(19) United States
(12) Patent Application Publication
(10) Pub. No.: US 2008/0096980 A1
(43) Pub. Date: Apr. 24, 2008

(54) Methods and Compositions for Reducing a Level of a Toxic Protein in a Cell

(76) Inventors: Steven M. Finkbeiner, Corte Madera, CA (US); Siddhartha Mitra, El Cerrito, CA (US); Montserrat Arrasate, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/572,980

(22) PCT Filed: Sep. 7, 2005
(86) PCT No.: PCT/US05/31881

§ 371 Date: Mar. 15, 2007

Related U.S. Application Data
(60) Provisional application No. 60/607,849, filed on Sep. 7, 2004.

(51) Int. Cl. A61K 47/00; A61P 25/00; C12N 5/06; C12Q 1/02

(52) U.S. Cl. 514/789; 435/29; 435/375; 435/69.1

(57) ABSTRACT

The present invention provides a method of reducing a level of a toxic protein in the cytosol of a cell, the method generally involving contacting the cell with an agent that induces a survival response by the cell. The present invention provides a method of reducing a level of a toxic protein in a cell, the method generally involving contacting the cell with an agent that induces sequestration of the toxic protein in an inclusion body in the cell. The present invention further provides methods of identifying an agent that induces sequestration of a toxic protein into an inclusion body. The present invention further provides methods of increasing the yield of a recombinant protein synthesized by a genetically modified prokaryotic or eukaryotic host cell. The present invention provides methods for treating disorders associated with the presence in a cell of a toxic protein, the methods generally involving administering to a subject having such a disorder an agent that induces a survival response in a cell that has the toxic protein, e.g., an agent that induces sequestration of the toxic protein in an inclusion body in the cell. The present invention further provides agents that induce a survival response by a cell; agents that induce sequestration of a toxic protein in an inclusion body in a cell; and compositions that include such agents.

### CROSS-REFERENCE

This application claims the benefit of U.S. Provisional Patent Application No. 60/607,849 filed Sep. 7, 2004, which application is incorporated herein by reference in its entirety.

### STATEMENT REGARDING FEDERALLY SPONSORED RESEARCH

The U.S. government may have certain rights in this invention, pursuant to grant nos. NS39074 and NS45191 awarded by the National Institutes of Health.

### FIELD OF THE INVENTION

The present invention is in the field of reducing levels of toxic proteins in cells, such as neuronal cells, to treat disorders associated with the presence of such toxic proteins.

### BACKGROUND OF THE INVENTION

The presence of toxic proteins in a cell is the cause of various pathologies, including Parkinson's Disease, Huntington's Disease, and Alzheimer's Disease. Alterations of protein conformation may result in the conversion of normally soluble and functional proteins into insoluble and pathogenic states. Examples of such insoluble proteins include: Beta-Amyloid Precursor Protein (APP) and Beta-Amyloid (βA) in amyloid plaques of Alzheimer's Disease (AD), Familial AD (FAD) and cerebral amyloid angiopathy (CAA); α-synuclein deposits in Lewy bodies of Parkinson's disease; Tau in neurofibrillary tangles in frontal temporal dementia and Pick's disease; Superoxide Dismutase in amyotrophic lateral sclerosis; abnormal Huntingtin protein in Huntington's disease; and Prion Protein (PrP) in Creutzfeldt-Jakob disease (CJD).

Huntington's disease is caused by an abnormal polyglutamine expansion within the protein huntingtin and is characterized by microscopic inclusion bodies of aggregated huntingtin and by the death of selected types of neuron. Huntington's Disease (HD) is a devastating neurological disease which usually presents in mid adult life and results in psychiatric disturbance, involuntary movement disorder, and cognitive decline associated with inexorable progression to death, typically 17 years following onset. HD is a progressive neurodegenerative disease striking principally medium spiny GABAergic neurons of the caudate nucleus of the basal ganglia. It affects about one in 10,000 individuals and is transmitted in an autosomal dominant fashion.

There is an ongoing need for methods of treating disorders associated with the presence of a toxic protein in a cell. The present invention addresses this need.

### Literature

Davies et al. (1997) Cell 90:537-548; Becher et al. (1998) Neurobiol. Dis. 4387-397; DiFiglia et al. (1997) Science 277:1990-1993; Ordway et al. (1997) Cell 91:753-763; Saudou et al. (1998) Cell 95:55-66; Klement et al. (1998) Cell 95:41-53; Cummings et al. (1999) Neuron 24:879-892; Taylor et al. (2003) Hum. Mol. Genet. 12:749-757; Shimohata et al. (2002) Neurosci. Lett. 323:215-218; Sisodia et al. (1998) Cell 95:1-4; Bence et al. (2001) Science 292:1552-1555; Ross (1997) Neuron 19:1147-1150; Preisinger et al. (1999) Philos. Trans. R. Soc. Lond. B. Biol. Sci. 354:1029-1034; Apostol et al. (2003) Proc. Natl. Acad. Sci. USA 100:5950-5955; Kuemmerle et al. (1999) Ann. Neurol 46:842-849; Gutekunst et al. (1999) J. Neurosci. 19:2522-2534; U.S. Pat. No. 6,743,771; U.S. Pat. No. 6,420,122.

### SUMMARY OF THE INVENTION

The present invention provides a method of reducing a level of a toxic protein in the cytosol of a cell, the method generally involving contacting the cell with an agent that induces a survival response by the cell. The present invention provides a method of reducing a level of a toxic protein in a cell, the method generally involving contacting the cell with an agent that induces sequestration of the toxic protein in an inclusion body in the cell. The present invention further provides methods of identifying an agent that induces sequestration of a toxic protein into an inclusion body. The present invention further provides methods of increasing the yield of a recombinant protein synthesized by a genetically modified prokaryotic or eukaryotic host cell. The present invention provides methods for treating disorders associated with the presence in a cell of a toxic protein, the methods generally involving administering to a subject having such a disorder an agent that induces a survival response in a cell that has the toxic protein, e.g., an agent that induces sequestration of the toxic protein in an inclusion body in the cell. The present invention further provides agents that induce a survival response by a cell; agents that induce sequestration of a toxic protein in an inclusion body in a cell; and compositions that include such agents.

### BRIEF DESCRIPTION OF THE DRAWINGS

FIGS. 1A-D depict polyQ expansion-dependent cell death measured with an automated microscope.

FIGS. 2A-E depict neuronal death without IB formation.

FIGS. 3A-F depicts the effect of diffuse mutant htt on neuronal survival.

FIGS. 4A-F depict the effect of IB formation on neuronal survival.

FIG. 5 depicts a model of the role of IB formation in huntingtin-induced neurodegeneration.

FIG. 6 depicts fluorescence of mRFP as a measure of neuronal morphology and viability that is independent of htt^{ex1}-GFP.

FIG. 7 depicts loss of fluorescence from transfected mRFP as a sensitive and specific assay of neuronal death.

FIG. 8 depicts polyQ expansion-dependent cell death measured with an automated microscope.

FIG. 9 depicts a comparison of single-neuron levels of GFP estimated by imaging with levels measured by immunocytochemistry.

FIG. 10 depicts survival of neurons with similar levels of htt^{ex1}-Q47-GFP that form IBs compared with survival of neurons that do not form IBs.

FIG. 11 depicts association of IB formation with reduced death risk and increased survival among neurons transfected with htt^{ex1}-47Q-GFP.

FIGS. 12A-C depict viability of neurons with IBs as assessed by annexin V staining.

FIG. 13 depicts the effect of IB formation on death risk and survival among stably transfected PC12 cells that were induced to express htt^{ex1}-103Q-GFP.

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, murines, simians, humans, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “an inclusion body” includes a plurality of such inclusion bodies and reference to “the active agent” includes reference to one or more active agents and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention is based in part on the observation that formation of inclusion bodies (IB) containing an abnormal and toxic form of huntingtin (htt) function to increase neuron survival. Huntington's disease (HD), a neurodegenerative disorder caused by an abnormal polyglutamine (polyQ) expansion within the htt protein, is characterized by aggregation of htt into microscopic intracellular deposits called inclusion bodies (IBs) and by the death of striatal and cortical neurons. However, the relationship between htt deposition and neurodegeneration is controversial. Some reports indicate that IB formation is associated with neurodegeneration. See, e.g., Davies et al. (1997) Cell 90:537-548; Becher et al. (1998) Neurobiol. Dis. 4387-397; DiFiglia et al. (1997) Science 277:1990-1993; and Ordway et al. (1997) Cell 91:753-763. However, other reports indicate no correlation, or a negative correlation, between IB formation and neurodegeneration. See, e.g., Saudou et al. (1998) Cell 95:55-66; Klement et al. (1998) Cell 95:41-53; Cummings et al. (1999) Neuron 24:879-892; Taylor et al. (2003) Hum. Mol. Genet. 12:749-757; and Shimohata et al. (2002) Neurosci. Lett. 323:215-218. Three competing models have described IB formation as pathogenic, incidental, or a beneficial coping response. See, e.g., Saudou et al. (1998) supra; Sisodia et al. (1998) Cell 95:1-4; Bence et al. (2001) Science 292:1552-1555; and Ross (1997) Neuron 19:1147-1150.

IBs seem to result from aggregation that generates many protein complexes that differ in multimerization and three-dimensional structure. Poirier et al. (2002) J. Biol. Chem. 277:41032-41037. These complexes often co-exist with IBs, but low temporal and spatial resolution limited interpretation of past experiments that correlated IB formation with neurodegeneration. Attempts to disrupt the aggregation process yielded opposite results, depending on the manipulation (Saudou et al. (1998) supra; Cummings et al. (1999) supra; Shimohata et al. (2002) supra; Chen et al. (2001) J. Mol. Biol. 311:173-182; Wyttenbach et al. (2000) Proc. Natl. Acad. Sci. USA 97:2898-2903; Muchowski et al. (2002) Proc. Natl. Acad. Sci. USA 99;727-732), probably because it is difficult if not impossible, using currently methodologies, to manipulate aggregation selectively. Perutz et al. (2001) Nature 412:143-144.

It has now been found that formation of IB containing an abnormal and toxic form of htt function to increase neuron survival. The present invention makes use of this observation, and provides methods of reducing levels of a toxic protein in a cell, by inducing sequestration of the toxic protein into an inclusion body.

The present invention provides a method of reducing a level of a toxic protein in a cell, the method generally involving contacting the cell with an agent that induces a survival response by the cell. The present invention provides a method of reducing a level of a toxic protein in a cell, the method generally involving contacting the cell with an agent that induces sequestration of the toxic protein in an inclusion body in the cell. The present invention provides methods for treating disorders associated with the presence in a cell of a toxic protein, the methods generally involving administering to a subject having such a disorder an agent that induces a survival response in a cell that has the toxic protein, e.g., an agent that induces sequestration of the toxic protein in an inclusion body in the cell. The present invention further provides agents that induce a survival response by a cell; agents that induce sequestration of a toxic protein into an inclusion body in a cell; and compositions that include such agents.

The present invention further provides methods of increasing the yield of a recombinant protein synthesized by a genetically modified prokaryotic or eukaryotic host cell. The methods generally involve culturing a host cell that is genetically modified with an expression vector comprising a nucleotide sequence encoding a protein of interest (a “recombinant protein”) in the presence of an agent that induces sequestration of the protein into an inclusion body in the host cell; and purifying the recombinant protein from the inclusion body.

The present invention further provides methods of treating cancer, or inhibiting growth of a cancerous cell, the methods generally involving inhibiting sequestration of a toxic protein, present in the cytosol of the cancerous cell, into an inclusion body. In instances in which a cancerous cell sequesters a toxic protein into an inclusion body, thereby rendering the toxic protein less toxic or non-toxic toward the cell, inhibiting sequestration of the toxic protein into an inclusion body increases the effective amount of toxic protein in the cell and/or enhances the toxicity of the protein toward the cancerous cell.

The present invention further provides methods of inhibiting sequestration of a toxic protein into an inclusion body in a prokaryotic cell, the method generally involving contacting the cell with an agent that inhibits sequestration of the toxic protein into an inclusion body in the prokaryotic cell. Such a method is useful, e.g., to effectively increase the level of soluble toxic protein in the prokaryotic cell, thereby killing the cell or reducing the growth of the cell. In these embodiments, therefore, an agent that inhibits sequestration of the toxic protein into an inclusion body in the prokaryotic cell is a bactericidal agent or a bacteriostatic agent.

### Methods of Inducing a Survival Response; Methods of reducing a Level of a Toxic Protein in a Eukaryotic Cell

The present invention provides a method of inducing a survival response in a eukaryotic host cell, wherein the eukaryotic cell is one that contains a toxic protein(s), e.g., in the cytosol of the cell. The method generally involves contacting the cell with an agent that induces a survival response in the eukaryotic cell. A survival response includes one or more of the following: 1) sequestration of a toxic protein from the cytosol into an inclusion body; 2) an increase in proteasome function; 3) an increase in chaperone protein activity; 4) suppression of apoptosis signals; and 5) mitotic arrest.

The present invention provides a method of reducing a level of a toxic protein in a eukaryotic cell, the method generally involving contacting the cell with an agent that induces a survival response by the cell. The present invention provides a method of reducing a level of a toxic protein in a cell, the method generally involving contacting the cell with an agent that induces sequestration of the toxic protein in an inclusion body in the cell.

Cells that contain a toxic protein, and that are amenable to treatment with a subject method, include eukaryotic cells of multicellular organisms, where multicellular organisms include, but are not limited to, mammals, reptiles, amphibians, avian species, plants, etc. Such cells include, but are not limited to, neuronal cells; virus-infected cells; cancerous cells of any cell type; muscle cell; spleen cells; T cell; B cells; dendritic cells; liver cells; kidney cells; epithelial cells; endothelial cells; etc. In some embodiments, the cell is a neuronal cell, e.g., a substantia nigra neuron, a hippocampal neuron, a trigeminal nerve neuron, a cerebellar neuron, a cerebral cortex neurons, a spinal cord neuron, and the like. In some embodiments, the cell is a neuroglial cell, e.g., an astrocyte, an oligodendrocyte, an ependymal cell, a microglial cell, etc.

An agent that induces sequestration of a toxic protein into an inclusion body is generally an agent that reduces the level of toxic protein in an area of the cell other than an inclusion body, e.g., in the cytosol of the cell. Thus, e.g., an agent that induces sequestration of a toxic protein into an inclusion body reduces the level of a toxic protein in an area of the cell other than an inclusion body (e.g., the cytosol) by at least about 10%, at least about 20%, at least about 30%, at least about 40%, at least about 50%, at least about 60%, at least about 70%, at least about 80%, or at least about 90%, or more, compared to the level of the toxic protein in an area of the cell other than an inclusion body (e.g., the cytosol) in the absence of the agent.

An agent that induces sequestration of a toxic protein into an inclusion body induces sequestration of at least about 20%, at least about 30%, at least about 40%, at least about 50%, at least about 60%, at least about 70%, at least about 80%, or at least about 90%, or more, of the total amount of the toxic protein present in the cell into inclusion bodies. Whether an agent induces sequestration of a toxic protein into an inclusion body in a cell is determined using any known method, including the microscopic analysis described in the Examples, infra.

Of particular interest in many embodiments are agents that reduce the level of a toxic protein in an area of the cell other than an inclusion body, such that no toxicity is detected. Methods of detecting toxicity are well known in the art. Toxicity results in some instances in cell death. Methods of detecting cell death are well known in the art and include, but are not limited to, an apoptosis assay; an assay for release of a detectable marker; an assay for binding of a dye to DNA; etc.

Apoptosis assays are well known in the art. Assays can be conducted on cell populations or an individual cell, and include morphological assays and biochemical assays. A non-limiting example of a method of determining the level of apoptosis in a cell or a cell population is TUNEL (TdT-mediated dUTP nick-end labeling) labeling of the 3′-OH free end of DNA fragments produced during apoptosis (Gavrieli et al. (1992) J. Cell Biol. 119:493). The TUNEL method involves catalytically adding a nucleotide, which has been conjugated to a chromogen system or a to a fluorescent tag, to the 3′-OH end of the 180-bp (base pair) oligomer DNA fragments in order to detect the fragments. The presence of a DNA ladder of 180-bp oligomers is indicative of apoptosis. Procedures to detect cell death based on the TUNEL method are available commercially, e.g., from Boehringer Mannheim (Cell Death Kit) and Oncor (Apoptag Plus). Another marker that is currently available is annexin, sold under the trademark APOPTEST™. This marker is used in the “Apoptosis Detection Kit,” which is also commercially available, e.g., from R&D Systems. During apoptosis, a cell membrane's phospholipid asymmetry changes such that the phospholipids are exposed on the outer membrane. Annexins are a homologous group of proteins that bind phospholipids in the presence of calcium. A second reagent, propidium iodide (PI), is a DNA binding fluorochrome. When a cell population is exposed to both reagents, apoptotic cells stain positive for annexin and negative for PI, necrotic cells stain positive for both, live cells stain negative for both. Other methods of testing for apoptosis are known in the art and can be used, including, e.g., the method disclosed in U.S. Pat. No. 6,048,703.

Detectable marker-release assays to detect cell death are well known in the art. For example, a cell containing a fluorescent dye is monitored for the loss of fluorescence, where loss of fluorescence is an indication of cell death. Detectable markers suitable for use include, but are not limited to, fluorescent dyes, fluorescent proteins, and the like. Suitable fluorescent proteins include, but are not limited to, a green fluorescent protein (GFP), including, but not limited to, a GFP derived from Aequoria victoria or a derivative thereof, e.g., a “humanized” derivative such as Enhanced GFP, which are available commercially, e.g., from Clontech, Inc.; a GFP from another species such as Renilla reniformis, Renilla mulleri, or Ptilosarcus guernyi, as described in, e.g., WO 99/49019 and Peelle et al. (2001) J. Protein Chem. 20:507-519; a red fluorescent protein; any of a variety of fluorescent and colored proteins from Anthozoan species, as described in, e.g., Matz et al. (1999) Nature Biotechnol. 17:969-973; and the like. Suitable detectable markers include dyes such as fluorescent dyes, e.g., coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum dye, etc.

DNA-binding dye assays to detect cell death are well known in the art, and include propidium iodide (PI) binding assay. PI enters dead, but not living, cells and binds to DNA within the cells. In some embodiments, binding of PI to cellular DNA is detected using flow cytometry. Flow cytometry devices and protocols are well known in the art, and have been amply described in numerous publications. See, e.g., Flow Cytometry and Sorting, 2^{nd} ed. (1990) M. R. Melamed et al., eds. Wiley-Liss; Flow Cytometry and Cell Sorting, 2^{nd} ed. (2000) A. Radbruch, Springer-Verlag; and In Living Color: Protocols in Flow Cytometry and Cell Sorting (2000) Diamond and Demaggio, eds, Springer-Verlag.

A Fluorescence energy transfer (FRET) assay can also be used to detect cell death. FRET is generated when green fluorescent protein (GFP) and blue fluorescent protein (BFP) are covalently linked together by a short peptide. Cleavage of this linkage by protease completely eliminates FRET effect. Caspase-3 (CPP32) is an important cellular protease activated during programmed cell death. An 18 amino acid peptide containing CPP32 recognition sequence, DEVD, is used to link GFP and BFP together. CPP32 activation is monitored by FRET assay during the apoptosis process. See, e.g., Xu et al. (1998) Nucleic Acids Res. 26 (8): 2034-2035.

The present invention provides a method of increasing survival of a cell that contains a toxic protein in the cytosol of the cell. The methods generally involve contacting a cell with an agent that induces sequestration of the toxic protein into an inclusion body in the cell. An agent that induces sequestration of the toxic protein into an inclusion body in the cell increases survival of the cell by at least about 10%, at least about 20%, at least about 30%, at least about 40%, at least about 50%, at least about 60%, at least about 70%, at least about 80%, or at least about 90%, or more, compared to the survival of the cell in the absence of the agent. For example, an agent that induces sequestration of the toxic protein into an inclusion body in the cell increases the proportion of a population of cells containing a toxic protein that survives by at least about 10%, at least about 20%, at least about 30%, at least about 40%, at least about 50%, at least about 60%, at least about 70%, at least about 80%, or at least about 90%, or more, e.g., at least 2-fold, at least 5-fold, at least 10-fold, or more, compared to the surviving proportion of the cell population containing a toxic protein in the absence of the agent.

The present invention provides a method of reducing the risk of death of a cell that contains a toxic protein in the cytosol of the cell. In the presence of an active agent that induces sequestration of a toxic protein into an inclusion body, the risk of death of the cell is reduced by at least about 20%, at least about 50%, at least about 80%, or more, compared to the risk of death of the cell in the absence of the agent.

### Toxic Proteins

Toxic proteins include any polypeptide that is toxic to a cell. In some cases, a protein is toxic only when present in the cell above a certain level or concentration. In such instances, the protein is present in the cell at a level that is toxic to the cell (a “toxic level”).

Such toxic proteins include, but are not limited to, polypeptides that contain an extended polyglutamine region (a “polyQ-containing toxic protein”); beta-amyloid polypeptides; tau proteins; presenilins; alpha-synucleins; and prion proteins. Examples of naturally occurring polypeptides that contain extended polyglutamine regions are abnormal forms of huntingtin, atropin-1, ataxin-1, ataxin-2, ataxin-3, ataxin-7, alpha 1A-voltage dependent calcium channel, and androgen receptor. Other toxic proteins include, but are not limited to, viral proteins (e.g., proteins encoded by herpes simplex virus, human immunodeficiency virus, cytomegalovirus, Epstein-Barr virus, etc.); certain tumor-associated proteins; a toxic protein present in an abnormal muscle cell, e.g., myopathies; a mis-folded protein that is cytotoxic; a mutant form of an otherwise normal or beneficial protein, where the mutant form of the protein is toxic to the cell; and the like. See, e.g., Laszlo et al. (1991) J. Pathol. 164:203-214; Lelouard et al. (2004) J. Cell Biol. 164:667-675; Lelouard et al. (2002) Nature 417:177-182; Dalakas (1992) Curr. Opin. Neurol. Neurosurg. 5:645-654; Glatzel et al. (2003) N. Engl. J. Med. 349:1812-1820.

Typically, the extended polyglutamine region in a polyQ-containing toxic protein includes from about one glutamine residue to about 3 glutamine residues, from about 3 glutamine residues to about 5 glutamine residues, from about 5 glutamine residues to about 10 glutamine residues, from about 10 glutamine residues to about 15 glutamine residues, from about 15 glutamine residues to about 20 glutamine residues, from about 20 glutamine residues to about 25 glutamine residues, from about 25 glutamine residues to about 30 glutamine residues, from about 30 glutamine residues to about 35 glutamine residues, from about 35 glutamine residues to about 40 glutamine residues, from about 40 glutamine residues to about 50 glutamine residues, or from about 50 glutamine residues to about 60 glutamine residues, or more, more than the number of glutamine residues in a polyglutamine region that is found in a normal, non-toxic protein. For example, the extended polyglutamine region in a toxic protein is generally from about 30 glutamine residues to about 40 glutamine residues, from about 40 glutamine residues to about 50 glutamine residues, from about 50 glutamine residues to about 60 glutamine residues, from about 60 glutamine residues to about 80 glutamine residues, from about 80 glutamine residues to about 80 glutamine residues, from about 80 glutamine residues to about 90 glutamine residues, from about 90 glutamine residues to about 100 glutamine residues, from about 100 glutamine residues to about 150 glutamine residues, from about 150 glutamine residues to about 200 glutamine residues, from about 200 glutamine residues to about 250 glutamine residues, or from about 250 glutamine residues to about 300 glutamine residues, or more, in length.

### Screening Methods

The present invention provides methods of identifying an agent that induces a survival response in a cell, e.g., an agent that induces sequestration of a toxic protein into an inclusion body. The methods generally involve contacting a cell that includes a toxic protein in the cytosol of the cell with a test agent; and determining the effect, if any, of the test agent on sequestration of the toxic protein into an inclusion body in the cell. In some embodiments, the methods further involve determining the effect, if any, of the test agent on cell viability.

The terms “candidate agent,” “test agent,” “agent,” “substance,” and “compound” are used interchangeably herein. Candidate agents encompass numerous chemical classes, typically synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. New potential therapeutic agents may also be created using methods such as rational drug design or computer modeling.

Candidate agents are generally small organic or inorganic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Screening may be directed to known pharmacologically active compounds and chemical analogs thereof, or to new agents with unknown properties such as those created through rational drug design.

As used herein, the term “determining” refers to both quantitative and qualitative determinations and as such, the term “determining” is used interchangeably herein with “assaying,” “measuring,” and the like.

Assays of the invention include controls, where suitable controls include a control cell (e.g., a cell of the same cell type or same tissue type) not administered with the test agent. In some embodiments, a plurality of assays is run in parallel with different agent concentrations to obtain a differential response to the various concentrations. Typically, one of these concentrations serves as a negative control, i.e. at zero concentration or below the level of detection.

Agents that have an effect in an assay method of the invention may be further tested for cytotoxicity, bioavailability, and the like, using well known assays. Agents that have an effect in an assay method of the invention may be subjected to directed or random and/or directed chemical modifications, such as acylation, alkylation, esterification, amidification, etc. to produce structural analogs. Such structural analogs include those that increase bioavailability, and/or reduced cytotoxicity. Those skilled in the art can readily envision and generate a wide variety of structural analogs, and test them for desired properties such as increased bioavailability and/or reduced cytotoxicity and/or ability to cross the blood-brain barrier.

Whether a test agent induces sequestration of a toxic protein into an inclusion body can be determined using any known method. In some embodiments, determination of whether a test agent induces sequestration of a toxic protein into an inclusion body is carried out using a microscopic imaging assay, as described in the Examples, infra.

In some embodiments, a subject screening method further comprises determining the effect, if any, of the test agent on viability of the cell. A candidate agent is assessed for any cytotoxic activity it may exhibit toward the cell used in the assay, using well-known assays, such as trypan blue dye exclusion, an MTT (3-(4,5-dimethylthiazol-2-yl)-2,5-diphenyl-2H-tetrazolium bromide) assay, and the like; or an assay as described in the Examples.

A test agent of interest is one that induces sequestration of at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, of a cytosolic toxic protein into an inclusion body, compared to the amount of the toxic protein in the cytosol in the absence of the test agent.

### Treatment Methods

The present invention provides methods of treating a disorder associated with the presence in a cell of a toxic protein. The methods generally involve administering to an individual in need thereof an effective amount of an active agent that induces sequestration of a toxic protein into an inclusion body in an affected cell in the individual.

Non-limiting examples of toxic proteins and their associated disorders are as follows: huntingtin, which is associated with Huntington's disease; atrophin-1, which is associated with dentatorubralpallidoluysian atrophy; ataxin-1, which is associated with spinocerebellar ataxia type 1; ataxin-2, which is associated with spinocerebellar ataxia type-2; ataxin-3, which is associated with spinocerebellar ataxia type-3; alpha 1a-voltage dependent calcium channel, which is associated with spinocerebellar ataxia type-6; ataxin-7, which is associated with spinocerebellar ataxia type-7; and androgen receptor, which is associated with spinobulber muscular atrophy; synuclein proteins, namely alpha, beta and gamma synucleins, which are associated with Alzheimer's disease, Parkinson's disease and breast cancer; amyloid light chains and amyloid-associated proteins, which are associated with amyloidosis; mutant transthyretin, which is associated with familial amyloid polyneuropathies; beta2 microglobulin, aggregation of which causes complications during chronic renal dialysis; beta amyloid protein, which is associated with Alzheimer's disease; immunoglobulin light chain, which is associated with multiple myelomas and various other B-cell proliferations; prion proteins, which cause spongiform encephalopathies such as Creutzfeldt-Jakob disease (CJD), variant CJD, Gerstmann-Sträussler-Scheinker disease, and kuru in humans, bovine spongieform encephalopathy in bovines, and scrapie in sheep and goats; viral proteins, produced during the course of a viral infection; misfolded proteins produced in an ischemic state, associated with stroke; etc.

Whether an active agent is effective in inducing sequestration of a toxic protein into an inclusion body, and in treating the disease or disorder associated with the presence in a cell in the individual of a toxic protein, can be readily determined. Whether an agent is effective in inducing sequestration of a toxic protein into an inclusion body is determined as described elsewhere herein. Whether an agent is effective n treating the disease or disorder associated with the presence in a cell in the individual of a toxic protein is readily determined by those of ordinary skill in the art, using standard methods for diagnosing the particular disease, and/or using standard methods for assessing the stage or extent of the disease, and/or using standard methods for assessing the severity of the disease or a symptom of the disease.

### Active Agents

The present invention provides active agents that induce sequestration of a toxic protein into an inclusion body in a eukaryotic cell, which agents thus increase survival of the cell. The terms “agent,” “active agent,” “substance,” “drug,” and “compound” are used interchangeably herein. Active agents include, but are not limited to, small molecule active agents, and peptide active agents.

### Small Molecule Active Agents

In some embodiments, an active agent that induces sequestration of a toxic protein into an inclusion body is a small molecule. Small molecule active agents include synthetic compounds, naturally-occurring compounds, fragments of naturally-occurring compounds; and the like. Small molecule active agents encompass numerous chemical classes, typically synthetic, semi-synthetic, or naturally-occurring inorganic or organic molecules. Small molecule active agents may be small organic compounds having a molecular weight of more than 50 and less than about 2,500 daltons. Small molecule active agents may comprise functional groups necessary for structural interaction with proteins, particularly hydrogen bonding, and may include at least an amine, carbonyl, hydroxyl or carboxyl group, and may contain at least two of the functional chemical groups. The agents may comprise cyclical carbon or heterocyclic structures and/or aromatic or polyaromatic structures substituted with one or more of the above functional groups. Small molecule active agents are also found among biomolecules including peptides, saccharides, fatty acids, steroids, purines, pyrimidines, derivatives, structural analogs or combinations thereof.

### Peptide Active Agents

In some embodiments, an active agent is a peptide. Suitable peptides include peptides of from about 3 amino acids to about 50 amino acids, from about 5 amino acids to about 30 amino acids, from about 10 amino acids to about 25 amino acids, from about 25 amino acids to about 50 amino acids, from about 50 amino acids to about 75 amino acids, or from about 75 amino acids to about 100 amino acids in length.

Peptides can include naturally-occurring and non-naturally occurring amino acids. Peptides may comprise D-amino acids, a combination of D- and L-amino acids, and various “designer” amino acids (e.g., P-methyl amino acids, Ca-methyl amino acids, and Nα-methyl amino acids, etc.) to convey special properties to peptides. Additionally, peptide may be a cyclic peptide. Peptides may include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3S)-methylphenylalanine, (2S,3R)-methyl-phenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; β-carboline (D and L); HIC (histidine isoquinoline carboxylic acid); and HIC (histidine cyclic urea). Amino acid analogs and peptidomimetics may be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; β-helix inducing analogs; γ-turn inducing analogs; Gly-Ala turn analog; amide bond isostere; tretrazol; and the like.

A peptide may be a depsipeptide, which may be a linear or a cyclic depsipeptide. Kuisle et al. (1999) Tet. Letters 40:1203-1206. “Depsipeptides” are compounds containing a sequence of at least two alpha-amino acids and at least one alpha-hydroxy carboxylic acid, which are bound through at least one normal peptide link and ester links, derived from the hydroxy carboxylic acids, where “linear depsipeptides” may comprise rings formed through S-bridges, or through an hydroxy or a mercapto group of an hydroxy-, or mercapto-amino acid and the carboxyl group of another amino- or hydroxy-acid but do not comprise rings formed only through peptide or ester links derived from hydroxy carboxylic acids. “Cyclic depsipeptides” are peptides containing at least one ring formed only through peptide or ester links, derived from hydroxy carboxylic acids.

Peptides may be cyclic or bicyclic. For example, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Methods for making cyclic peptides are well known in the art.

The term “bicyclic” refers to a peptide in which there exists two ring closures. The ring closures are formed by covalent linkages between amino acids in the peptide. A covalent linkage between two nonadjacent amino acids constitutes a ring closure, as does a second covalent linkage between a pair of adjacent amino acids which are already linked by a covalent peptide linkage. The covalent linkages forming the ring closures may be amide linkages, i.e., the linkage formed between a free amino on one amino acid and a free carboxyl of a second amino acid, or linkages formed between the side chains or “R” groups of amino acids in the peptides. Thus, bicyclic peptides may be “true” bicyclic peptides, i.e., peptides cyclized by the formation of a peptide bond between the N-terminus and the C-terminus of the peptide, or they may be “depsi-bicyclic” peptides, i.e., peptides in which the terminal amino acids are covalently linked through their side chain moieties.

A desamino or descarboxy residue can be incorporated at the terminii of the peptide, so that there is no terminal amino or carboxyl group, to decrease susceptibility to proteases or to restrict the conformation of the peptide. C-terminal functional groups include amide, amide lower alkyl, amide di(lower alkyl), lower alkoxy, hydroxy, and carboxy, and the lower ester derivatives thereof, and the pharmaceutically acceptable salts thereof.

In addition to the foregoing N-terminal and C-terminal modifications, a peptide or peptidomimetic can be modified with or covalently coupled to one or more of a variety of hydrophilic polymers to increase solubility and circulation half-life of the peptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky, S., Bioconjugate Chem., 6:150-165 (1995); Monfardini, C, et al., Bioconjugate Chem., 6:62-69 (1995); U.S. Pat. Nos. 4,640,835; 4,496,689; 4,3015144; 4,670,417; 4,791,192; 4,179,337 or WO 95/34326.

An active peptide will in some embodiments be conjugated to decapeptides comprised of Arginine residues to allow uptake across the plasma membrane by protein transduction. Such modifications allow peptides to enter cells (e.g., cross the plasma membrane) with high efficiency.

In some embodiments, an active peptide is a peptide aptamer. Peptide aptamers are peptides or small polypeptides that act as dominant inhibitors of protein function. Peptide aptamers specifically bind to target proteins, blocking their function ability. Kolonin and Finley (1998) Proc. Natl. Acad. Sci. USA 95:14266-14271. Due to the highly selective nature of peptide aptamers, they may be used not only to target a specific protein, but also to target specific functions of a given protein (e.g. a protein binding function). Further, peptide aptamers may be expressed in a controlled fashion by use of promoters which regulate expression in a temporal, spatial or inducible manner.

Peptide aptamers that bind with high affinity and specificity to a target protein may be isolated by a variety of techniques known in the art. Peptide aptamers can be isolated from random peptide libraries by yeast two-hybrid screens (Xu et al., (1997) Proc. Natl. Acad. Sci. USA 94:12473-12478). They can also be isolated from phage libraries (Hoogenboom et al, Immunotechnology (1998) 4:1-20) or chemically generated peptides/libraries.

The peptide active agent can be used in the form of the free peptide or a pharmaceutically acceptable salt. Amine salts can be prepared by mixing the peptide with an acid according to known methods. Suitable acids include inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalenesulfonic acid, and sulfanilic acid.

The peptides can generally be prepared following known techniques, as described for example in the cited publications, the teachings of which are specifically incorporated herein. In some embodiments, the peptides are prepared following the solid-phase synthetic technique initially described by Merrifield in J. Amer. Chem. Soc., 85, 2149-2154 (1963). Other techniques may be found, for example, in M. Bodanszky, et al., Peptide Synthesis, second edition, (John Wiley & Sons, 1976), as well as in other reference works known to those skilled in the art.

The peptides can also be prepared using standard genetic engineering techniques known to those skilled in the art. For example, the peptide can be produced by inserting nucleic acid encoding the peptide into an expression vector, expressing the DNA, and translating the RNA into the peptide in the presence of the required amino acids. The peptide is then purified using chromatographic or electrophoretic techniques, or by means of a carrier protein which can be fused to, and subsequently cleaved from, the peptide by inserting into the expression vector in phase with the peptide encoding sequence a nucleic acid sequence encoding the carrier protein. The fusion protein-peptide may be isolated using chromatographic, electrophoretic or immunological techniques (such as binding to a resin via an antibody to the carrier protein). The peptide can be cleaved using chemical methodology or enzymatically, as by, for example, hydrolases.

### Formulations, Dosages, Routes of Administration

An active agent is administered to an individual in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, an active agent may be administered to the host using any convenient means capable of resulting in the desired therapeutic effect. Thus, the agents can be incorporated into a variety of formulations for therapeutic administration. More particularly, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of an active agent(s) can be achieved in various ways, including oral, buccal, rectal, parenteral, intraperitoneal, intradermal, intrathecal, intraspinal, intracistemal, intracapsular, subcutaneous, intravenous, intramuscular, transdermal, intratracheal, etc., administration. In some embodiments, e.g., where two different agents are administered, two different routes of administration are used. Where the active agent is to be provided parenterally, such as by intravenous, subcutaneous, ophthalmic, intraperitoneal, intramuscular, vaginal, intraorbital, intracerebral, intracranial, intraspinal, intraventricular, intrathecal, intracisternal, intracapsular, intranasal or by aerosol administration, the agent typically comprises part of an aqueous or physiologically compatible fluid suspension or solution.

A liquid is in some embodiments the dosage form that is used for intravenous, intrathecal, intraspinal, intraventricular, or intramedullary administration of an active agent for treating spinal cord injuries. For preparing liquids, solvents can be used, as exemplified by purified water, physiological saline, alcohols such as ethanol, propylene glycol, glycerin and polyethylene glycol, and triacetin. The thus prepared liquids may be used as dilutions with a lactated Ringer's solution, a maintaining solution, a postoperative recovery fluid, a solution for supplying water to compensate for dehydration, physiological saline for use in dripping. The preparations may further be admixed with adjuvants such as antiseptics, moistening agents, emulsifiers, dispersing agents and stabilizers. Suspensions are another exemplary dosage form to be administered.

In some embodiments, an active agent is administered intrathecally, including, e.g., administration into a cerebral ventricle, administration into the lumbar area, and administration into the cisterna magna; or by an intraspinal route. For specific delivery within the central nervous system (CNS) intrathecal delivery can be used with, for example, an Ommaya reservoir. U.S. Pat. No. 5,455,044 provides for use of a dispersion system for CNS delivery or see U.S. Pat. No. 5,558,852 for a discussion of CNS delivery.

As used herein, the term “intrathecal administration” includes delivering an active agent directly into the cerebrospinal fluid of a subject, by techniques including lateral cerebroventricular injection through a burrhole or cisternal or lumbar puncture or the like (e.g., as described in Lazorthes et al. Advances in Drug Delivery Systems and Applications in Neurosurgery, 143-192 and Omaya et al., Cancer Drug Delivery, 1: 169-179, the contents of which are incorporated herein by reference). The term “lumbar region” includes the area between the third and fourth lumbar (lower back) vertebrae. The term “cisterna magna” includes the area where the skull ends and the spinal cord begins at the back of the head. The term “cerebral ventricle” includes the cavities in the brain that are continuous with the central canal of the spinal cord. Administration of an active agent to any of the above mentioned sites can be achieved by direct injection of the active agent or by the use of infusion pumps. For injection, the active agent can be formulated in liquid solutions, preferably in physiologically compatible buffers such as Hank's solution or Ringer's solution. In addition, the active agent may be formulated in solid form and re-dissolved or suspended immediately prior to use. Lyophilized forms are also included. The injection can be, for example, in the form of a bolus injection or continuous infusion (e.g., using infusion pumps) of the active agent.

Subcutaneous administration of an active agent can be accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of an active agent to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system.

In some embodiments, an active agent is delivered by a continuous delivery system. The terms “continuous delivery system,” “controlled delivery system,” and “controlled drug delivery device,” are used interchangeably to refer to controlled drug delivery devices, and encompass pumps in combination with catheters, injection devices, and the like, a wide variety of which are known in the art.

Mechanical or electromechanical infusion pumps are also suitable for use. Examples of such devices include those described in, for example, U.S. Pat. Nos. 4,692,147; 4,360,019; 4,487,603; 4,360,019; 4,725,852; 5,820,589; 5,643,207; 6,198,966; and the like. In some embodiments, drug delivery is accomplished using any of a variety of refillable, pump systems. Pumps provide consistent, controlled release over time. Typically, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are used in some embodiments because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for delivery of an active agent. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Pumps and other convective systems are used in some instances in view of their generally more consistent, controlled release over time. Osmotic pumps are used in some embodiments in view of their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, intraspinal, or other suitable site within a subject's body.

In some embodiments, an active agent is delivered using an implantable drug delivery system, e.g., a system that is programmable to provide for administration of a therapeutic agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the SynchroMed® infusion pump (Medtronic).

In pharmaceutical dosage forms, an active agent may be administered in the form of their pharmaceutically acceptable salts, or they may also be used alone or in appropriate association, as well as in combination, with other pharmaceutically active compounds. The following methods and excipients are merely exemplary and are in no way limiting.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, an active agent is formulated alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives, and flavoring agents.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the agent(s) in a composition as a solution in sterile water, normal saline or another pharmaceutically acceptable carrier.

### Crossing the Blood-Brain Barrier

The blood-brain barrier limits the uptake of many therapeutic agents into the brain and spinal cord from the general circulation. Molecules which cross the blood-brain barrier use two main mechanisms: free diffusion; and facilitated transport. Because of the presence of the blood-brain barrier, attaining beneficial concentrations of a given therapeutic agent in the central nervous system (CNS) may require the use of drug delivery strategies. Delivery of therapeutic agents to the CNS can be achieved by several methods.

One method relies on neurosurgical techniques. In the case of gravely ill patients such as accident victims or those suffering from various forms of dementia, surgical intervention is warranted despite its attendant risks. For instance, therapeutic agents can be delivered by direct physical introduction into the CNS, such as intraventricular or intrathecal injection of drugs. Intraventricular injection may be facilitated by an intraventricular catheter, for example, attached to a reservoir, such as an Ommaya reservoir. Methods of introduction may also be provided by rechargeable or biodegradable devices. Another approach is the disruption of the blood-brain barrier by substances which increase the permeability of the blood-brain barrier. Examples include intra-arterial infusion of poorly diffusible agents such as mannitol, pharmaceuticals which increase cerebrovascular permeability such as etoposide, or vasoactive agents such as leukotrienes. Neuwelt and Rappoport (1984) Fed. Proc. 43:214-219; Baba et al. (1991) J. Cereb. Blood Flow Metab. 11:638-643; and Gennuso et al. (1993) Cancer Invest. 11:638-643.

Further, it may be desirable to administer the pharmaceutical agents locally to the area in need of treatment; this may be achieved by, for example, local infusion during surgery, by injection, by means of a catheter, or by means of an implant, said implant being of a porous, non-porous, or gelatinous material, including membranes, such as silastic membranes, or fibers.

Therapeutic compounds can also be delivered by using pharmacological techniques including chemical modification or screening for an analog which will cross the blood-brain barrier. The compound may be modified to increase the hydrophobicity of the molecule, decrease net charge or molecular weight of the molecule, or modify the molecule, so that it will resemble one normally transported across the blood-brain barrier. Levin (1980) J. Med. Chem. 23:682-684; Pardridge (1991) in: Peptide Drug Delivery to the Brain; and Kostis et al. (1994) J. Clin. Pharmacol. 34:989-996.

Encapsulation of the drug in a hydrophobic environment such as liposomes is also effective in delivering drugs to the CNS. For example WO 91/04014 describes a liposomal delivery system in which the drug is encapsulated within liposomes to which molecules have been added that are normally transported across the blood-brain barrier.

Another method of formulating the drug to pass through the blood-brain barrier is to encapsulate the drug in a cyclodextrin. Any suitable cyclodextrin which passes through the blood-brain barrier may be employed, including, but not limited to, α-cyclodextrin, β-cyclodextrin and derivatives thereof. See generally, U.S. Pat. Nos. 5,017,566, 5,002,935 and 4,983,586. Such compositions may also include a glycerol derivative as described by U.S. Pat. No. 5,153,179.

Delivery may also be obtained by conjugation of a therapeutic agent to a transportable agent to yield a new chimeric transportable therapeutic agent. For example, vasoactive intestinal peptide analog (VIPa) exerted its vasoactive effects only after conjugation to a monoclonal antibody (mAb) to the specific carrier molecule transferrin receptor, which facilitated the uptake of the VIPa-mAb conjugate through the blood-brain barrier. Pardridge (1991); and Bickel et al. (1993) Proc. Natl. Acad Sci. USA 90:2618-2622. Several other specific transport systems have been identified, these include, but are not limited to, those for transferring insulin, or insulin-like growth factors I and II. Other suitable, non-specific carriers include, but are not limited to, pyridinium, fatty acids, inositol, cholesterol, and glucose derivatives. Certain prodrugs have been described whereby, upon entering the central nervous system, the drug is cleaved from the carrier to release the active drug. U.S. Pat. No. 5,017,566.

### Combination Therapies

In some embodiments, an active agent is administered in a combination therapy with at least one additional therapeutic agent. The at least one additional therapeutic agent will depend on the disorder or disease being treated. For example, current therapeutic agents used to treat Huntington's Disease (e.g., to reducing symptoms, preventing complications), include antipsychotics such as haloperidol, chlorpromazine, and olanzapine; antidepressants such as fluoxetine, sertraline hydrochloride, and nortriptyline; tranquilizers such as benzodiazepines, paroxetine, venlafaxin, and beta-blockers; mood-stabilizers such as lithium, valproate, and carbamazepine; and botulinum toxin.

### Dosages

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the a unit dosage forms of the present invention depend on the particular compound employed and the effect to be achieved, and the pharmacodynamics associated with each compound in the host.

### Dosages

The amount of an active agent which is administered will vary with the nature of the drug. As one non-limiting example, an active agent can be administered in the range of about 0.2 mg/kg/day to about 20 mg/kg/day. The determination of how large a dose is to be used may be determined using an animal model (e.g., a non-human primate model) and relating the dosage based on pharmacokinetics, e.g. with equations predictive of interspecies scaling. Usually, the lowest effective dose will be used.

In some embodiments, a single dose of an active agent is administered. In other embodiments, multiple doses of an active agent are administered. Where multiple doses are administered over a period of time, an active agent is administered twice daily (qid), daily (qd), every other day (qod), every third day, three times per week (tiw), or twice per week (biw) over a period of time. For example, an active agent is administered qid, qd, qod, tiw, or biw over a period of from one day to about 2 years or more. For example, an active agent is administered at any of the aforementioned frequencies for one week, two weeks, one month, two months, six months, one year, or two years, or more, depending on various factors.

Generally, unit dosage forms of an active agent range from about 1 μg to about 500 mg, e.g., from about 1 μg to about 5 μg, from about 5 μg to about 10 μg, from about 10 μg to about 25 μg, from about 25 μg to about 50 μg, from about 50 μg to about 75 μg, from about 75 μg to about 100 μg, from about 100 μg to about 125 μg, from about 125 μg to about 150 μg, from about 150 μg to about 200 μg, from about 200 μg to about 225 μg, from about 225 μg to about 250 μg, from about 250 μg to about 275 μg from about 275 μg to about 300 μg, from about 300 μg to about 400 μg, from about 400 μg to about 500 μg, from about 500 μg to about 600 μg, from about 600 μg to about 700 μg, from about 700 μg to about 800 μg, from about 800 μg to about 900 μg from about 900 μg to about 1000 μg, from about 1 mg to about 100 mg, from about 100 mg to about 200 mg, or from about 200 mg to about 500 mg.

An active agent can be administered twice daily, daily, every other day, once a week, twice a week, three times a week, every other week, three times per month, or once monthly, or substantially continuously or continuously.

An active agent is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time.

### Subjects Suitable For Treatment

Subjects suitable for treatment with a subject method, e.g., involving administering to the subject an effective amount of an agent that induces sequestration of a toxic protein into an inclusion body, include subjects suffering from any disorder or disease associated with the presence in a cell of a toxic protein. Such disorders include, but are not limited to, Huntington's disease; dentatorubralpallidoluysian atrophy; spinocerebellar ataxia type 1; spinocerebellar ataxia type-2; ataxin spinocerebellar ataxia type-3; spinocerebellar ataxia type-6; spinocerebellar ataxia type-7; spinobulber muscular atrophy; Alzheimer's disease; Parkinson's disease; amyloidosis; and spongiform encephalopathies such as Creutzfeldt-Jakob disease, kuru, variant CJD, and Gerstmann-Sträussler-Scheinker disease in humans, bovine spongieform encephalopathy in bovines, and scrapie in sheep and goats; viral infections; stroke; myopathies; cancer; etc.

### Increasing Production of Recombinantly Produced Proteins

The present invention provides methods of producing a recombinant protein in a genetically modified host cell. The methods generally involve contacting a genetically modified host cell that synthesizes a recombinant protein with an agent that induces sequestration of the recombinant protein into an inclusion body in the host cell; and isolating the recombinant protein from the inclusion body. The subject methods result in enhanced yield of the recombinant protein. The recombinant protein is also referred to herein as the “protein of interest.”

Using a subject method for producing a recombinant protein, the yield of recombinant protein is in excess of 5 mg per gram of wet weight of host cells. For example, a subject method yields from about 6 mg recombinant protein to about 10 mg recombinant protein, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, or from about 45 mg to about 50 mg, or more, per gram wet weight E. coli.

Genetically modified host cells are in many embodiments unicellular organisms, or are grown in culture as single cells. In some embodiments, the host cell is a eukaryotic cell. Suitable eukaryotic host cells include, but are not limited to, yeast cells, insect cells, plant cells, fungal cells, and algal cells. Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichiapyperi, Pichia stiptis, Pichia methanolica, Pichia sp., Saccharomyces cerevisiae, Saccharomyces sp., Hansenulapolymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, and the like.

In other embodiments, the genetically modified host cell is a prokaryotic cell. Suitable prokaryotic cells include, but are not limited to, any of a variety of laboratory strains of Escherichia coli, Lactobacillus sp., and the like.

Typically, an expression vector that includes a coding region encoding the protein of interest is introduced into the host cell, forming a genetically modified host cell. The expression vector will provide a transcriptional and translational initiation region, which may be inducible or constitutive, where the coding region is operably linked under the transcriptional control of the transcriptional initiation region, and a transcriptional and translational termination region. These control regions may be native to the coding region for the protein of interest, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins comprising the protein of interest fused to a fusion partner. Suitable fusion partners include, but are not limited to, a detectable enzyme, e.g., an enzyme such as β-galactosidase, luciferase, horse radish peroxidase, alkaline phosphatase, etc.; a detectable protein such as a red fluorescent protein, a greed fluorescent protein (GFP) derived from Aequoria victoria or a derivative thereof; a GFP from another species such as Renilla reniformis, Renilla mulleri, or Ptilosarcus guernyi, as described in, e.g., WO 99/49019 and Peelle et al. (2001) J. Protein Chem. 20:507-519; any of a variety of fluorescent and colored proteins from Anthozoan species, as described in, e.g., Matz et al. (1999) Nature Biotechnol. 17:969-973; and the like); peptides and polypeptides that confer enhanced stability in vivo (e.g., enhanced serum half-life); fusion partners that provide for ease of purification, e.g., (His)ₙ, e.g., 6His; an epitope tag, e.g., glutathione-S-transferase, hemagglutinin, FLAG, c-myc, and the like; a fusion partner that provides for multimerization, e.g., a multimerization domain such as an Fc portion of an immunoglobulin; and the like.

Expression cassettes may be prepared comprising a transcription initiation region, the coding region for the protein of interest, and a transcriptional termination region. Transcriptional control regions include promoters that provide for over-expression of the protein of interest in the genetically modified host cell; control regions that provide for inducible expression, such that when an inducing agent is added to the culture medium, transcription of the coding region of the protein of interest is induced.

Proteins and polypeptides may be expressed in prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g. COS 7 cells, may be used as the expression host cells.

In some situations, it is desirable to express the gene in eukaryotic cells, where the encoded protein will benefit from native folding and post-translational modifications. Small peptides can also be synthesized in the laboratory. Polypeptides that are subsets of the complete sequences of the subject proteins may be used to identify and investigate parts of the protein important for function.

Specific expression systems of interest include bacterial, yeast, insect cell and mammalian cell derived expression systems. Representative systems from each of these categories is are provided below:

Bacteria. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al, Proc. Natl. Acad. Sci (USA) (1983) 80:21-25; and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985) 25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990) 8:135; Kunze et al., J. Basic Microbiol. (1985) 25:141; Cregg et al., Mol. Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilburn et al., Gene (1983) 26:205-221; Yelton et al., Proc. Natl. Acad. Sci. (USA) (1984) 81:1470-1474; Kelly and Hynes, EMBO J. (1985) 4:475-479; EP 0 244,234; and WO 91/00357.

Insect Cells. Expression of heterologous genes in insects is accomplished as described in U.S. Pat. No. 4,745,051; Friesen et al;, “The Regulation of Baculovirus Gene Expression”, in: The Molecular Biology Of Baculoviruses (1986) (W. Doerfier, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Maeda et al., Nature (1985) 315:592-594.

Mammalian Cells. Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. RE 30,985.

The genetically modified host cell is contacted with an agent that induces sequestration of the protein of interest into an inclusion body in the host cell. The genetically modified host cell will in some embodiments be cultured in medium that includes an agent that induces sequestration of the protein of interest into an inclusion body. After a suitable period of time, the protein is isolated from the host cell. Any convenient protein purification procedures may be employed, where suitable protein purification methodologies are described in Guide to Protein Purification, (Deuthser ed.) (Academic Press, 1990); and Current Protocols in Protein Science, J. Coligan et al., eds. (2004) Wiley InterScience.

In many embodiments, purification of a protein of interest from the genetically modified host cell involves harvesting the cells by centrifugation; sonicating the cells; separating inclusion bodies from the remainder of the cells by low-speed centrifugation; contacting the inclusion bodies with detergent and a denaturant for a period of time to remove cell wall and outer membrane components; followed by solubilizing and re-folding the proteins by infusion of a redox solution, such as a buffer containing an oxidizing agent and a reducing agent; or followed by solubilizing the protein, purifying the protein in the presence of a denaturant, then re-folding the protein. One or more further purification steps can be carried out before or after the re-folding step.

Any standard purification method is suitable, including, but not limited to, high performance liquid chromatography, size exclusion chromatography, gel electrophoresis, affinity chromatography, and the like.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Effect of Inclusion Body Formation on Huntingtin Levels and Neuronal Survival

### Methods

### Plasmids

Expression plasmids encoding an N-terminal fragment of huntingtin (htt) fused to a green fluorescent protein (GFP) (pGW1-htt^{ex1}-[25, 47, 72, or 103Q]-GFP) were derived from pcDNA3.1-based plasmids (Kazantsev et al. (1999) Proc. Natl. Acad. Sci. USA 96:11404-11409) by subcloning into pGW1-CMV (British Biotechnologies). A polymerase chain reaction (PCR) product of exon 1 of human htt with 17 CAG repeats was ligated to GFP and used to create pGW1-htt^{ex1}-17Q-GFP. A PCR product of mRFP was ligated into pGW1-CMV to create pGW1-mRFP and into pcDNA3.1(30 ) to create pcDNA3.1-mRFP. Plasmid constructions were confirmed by DNA sequencing.

Cell Culture and Transfection

Primary cultures of rat striatal neurons were prepared from embryos (E16-18) and transfected with plasmids (6-7DIV) as described (Saudou et al. (1998) Cell 95:55-66; Finkbeiner et al. (1997) Neuron 19:1031-1047; and see, e.g., the following internet site: //gweb1.ucsf.edu/labs/finkbeiner). Typically, neurons were co-transfected with pGW1-mRFP and a version of pGW1-htt^{ex1}-[17, 25, 47, 72, or 103Q]-GFP in a 1:1 molar ratio, using a total of 1-4 μg of DNA in each well of a 24-well plate. After transfection, neurons were maintained in serum-free medium.

To perform a modified LIVE-DEAD assay, growth medium was replaced with Eagle basal medium 48 h after transfection. Twenty min before kainate (Sigma) treatment, ethidium homodimer (5 μM, Molecular Probes) was added, and images of transfected neurons were collected before and every 30 min after kainate addition. A detergent-resistance assay was performed as described (Kazantsev et al. (1999) Proc. Natl. Acad. Sci. USA 96:11404-11409) with minor modifications. Neurons with putative IBs were imaged, treated with 1% paraformaldehyde for 15 min at 37° C. followed by 5% Triton-X-100 and 5% SDS for 20 min at 37° C., and imaged again. PC12 cells inducibly expressing htt^{ex1}-25Q-GFP or htt^{ex1}-103Q-GFP⁵⁰ were plated at 1×10⁴ cells/cm², transiently transfected with pcDNA3.1-mRFP, and induced with 1 μM tebufenozide. In some experiments, wild-type PC12 cells were plated at 5×10⁴ cells/cm² and co-transfected with a version of pGW1-htt^{ex1}-[17, 25, 47, 72, or 103Q]-GFP and pcDNA3.1 mRFP in a 1:1 molar ratio using a total of 2 μg of DNA in each well of a 24-well plate.

### Immunocytochemistry

Striatal neurons grown on 12-mm glass coverslips were examined 36 h after transfection as described (Saudou et al. (1998), supra) with anti-GFP (1:500, Chemicon), anti-htt EM48 (1:50, Chemicon), and anti-chicken or anti-rabbit Cy3-labeled antibodies (1:300, Jackson Inmunochemical).

### Western Blots

HEK 293 cells grown in Dulbecco's modified Eagle medium with 10% calf serum, 2 mM glutamine, and penicillin/streptomycin were transiently transfected with pGW1-GFP (1-6 μg/well). Images were captured every 24 h for 3 days. Protein extracts were prepared from cells immediately after imaging, subjected to SDS-PAGE, blotted with anti-GFP antibody (1:1000; Zymed), and detected with ¹²⁵I-labeled secondary antibody and a phosphorimager screen (Fuji).

### Robotic Microscope Imaging System

The system is based on an inverted Nikon microscope (TE300 Quantum). Olympus 4× (N.A. 0.13) and 10× (N.A. 0.30) and Nikon 20× (N.A. 0.45) objectives were used. Xenon lamp (175 W) illumination was supplied by a liquid light guide to reduce electrical noise. Images were detected and digitized with a Hammatsu Orca II 12/14 bit, digital, cooled charge-coupled device (CCD) camera and Universal Imaging Metamorph software. Stage movements and focusing were executed with computer-controlled stepper motors. Fluorescence excitation and emission filters were moved into or out of the optical path with each program loop by two 10-position filter wheels (Sutter Instruments) under computer control. The whole system is mounted on a vibration isolation table to reduce noise. Computer commands that perform and coordinate automated stage movements, filter wheel movements, and focusing were generated with software programs that combine custom-designed and commercially available algorithms. Additional programs for image analysis were written with Mat Lab and Visual C software.

### Image and Statistical Analysis

Measurements of htt expression, inclusion body (IB) formation, and survival were extracted from files generated with automated imaging by automated analysis programs or by visual inspection. Automated programs identified living transfected neurons by physical dimensions and fluorescence. IBs were monitored by size and fluorescence intensity. Expression levels of GFP-tagged versions of htt were estimated by measuring GFP fluorescence intensity over a region of interest that corresponded to the cell soma or as otherwise indicated, using the fluorescence of co-transfected monomeric red fluorescent protein (mRFP) as a guide. These GFP intensity values are background subtracted using an adjacent area of the image.

For statistical analysis, survival time was defined as the imaging time point at which a cell was last seen alive. Kaplan-Meier curves were used to estimate survival and hazard functions with commercially available software (Statview). Differences in Kaplan-Meier curves were assessed with the log-rank test. Linear regression was used to correlate htt expression measured with different methods, and correlations between htt expression and survival or IB formation were made with Cox proportional hazard analysis. Differences in mean measurements were compared by ANOVA or t-test.

### Results

### Automated Microscopy of an HD Model

To increase the temporal resolution of conventional approaches, an automated microscope system was developed (Arrasate et al. (2003) Soc. Neurosci. 29:209.8; and U.S. Patent Publication No. 20030103662) that returns to precisely the same neuron or field of neurons, even after cells have been removed from the microscope stage during the interval. The survival of individual neurons, the intracellular levels of mutant htt, and aggregation of htt into IBs, were prospectively measured. The relationships among these factors were determined by survival analysis without introducing potentially confounding nonspecific manipulations. Collett, D. Modeling survival data in medical research (ed. Collett, D.) (Chapman & Hall, London, 1994); and Therneau, T. M. Modeling survival data: Extending the Cox model (eds. Theneau, T. M. & Grambsch, P. M.) (Springer, N.Y., 2000).

An established neuronal HD model (Saudou et al. (1998), supra), in which striatal neurons are transiently transfected with htt, was examined. The model recapitulates several HD features (e.g., IB formation and polyQ expansion-dependent, neuron-specific death). Saudou et al. (1998), supra. To visualize htt in living striatal neurons, we used N-terminal exon 1 fragments of htt (htt^{ex1}) containing polyQ stretches of various lengths and fused to the N-terminus of green fluorescent protein (GFP). Kazantsev et al. (1999) Proc. Natl. Acad. Sci. USA 96:11404-11409. A similar fragment may be generated in HD by proteolytic cleavage (Goldberg et al. (1996) Nat. Genet. 13:442-449; Scherzinger et al. (1997) Cell 90:549-558; Wellington et al. (1998) J. Biol. Chem. 273:9158-9167; Kim et al. (2001) Proc. Natl. Acad. Sci. USA 98:12784-12789; and Mende-Mueller et al. (2001) J. Neurosci. 21:1830-1837) and is sufficient to produce HD-like features when expressed as a transgene in a mouse. Mangiarini et al. (1996) Cell 87:493-506. Along with htt^{ex1}-GFP, neurons were co-transfected with a monomeric red fluorescent protein (mRFP) (Campbell et al. (2002) Proc. Natl. Acad. Sci. USA 99:7877-7882) to visualize neurons independently of htt^{ex1}-GFP (FIG. 6). Fluorescent protein expression and periodic imaging did not affect neuronal viability. Arrasate et al. (2003) supra.

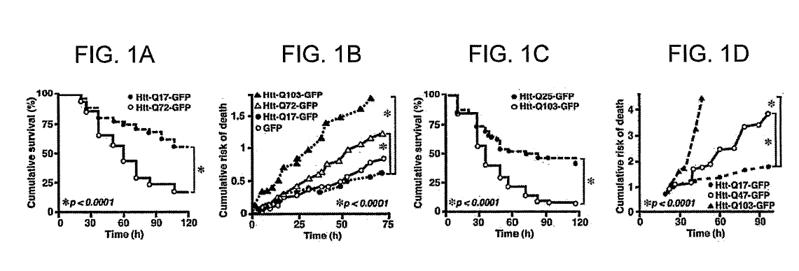

Neurons were imaged with the automated microscope 2-24 h after transfection and at 12-24-h intervals. Some neurons abruptly lost mRFP fluorescence. This event corresponded to the loss of membrane integrity and cell death and correlated well with other cell-death markers (FIG. 7). Others have found the loss of a fluorescent marker protein to be a highly sensitive and specific assay of cell death via different pathways and in different types of cell. Strebel et al. (2001) Cytometry 43:126-133. The ability to monitor individual neurons over time allows us to quantify differences in their longevity with survival analysis. Collett, D. Modeling survival data in medical research (ed. Collett, D.) (Chapman & Hall, London, 1994); and Therneau, T. M. Modeling survival data: Extending the Cox model (eds. Therneau, T. M. & Grambsch, P. M.) (Springer, N.Y., 2000). The survival function for neurons transfected with GFP or with htt^{ex1}-GFP containing a normal (17Q) or an expanded (72Q) polyQ stretch was determined. Neurons transfected with htt^{ex1}-GFP containing disease-associated polyQ stretches died faster than neurons transfected with htt^{ex1}-GFP (FIG. 1A).

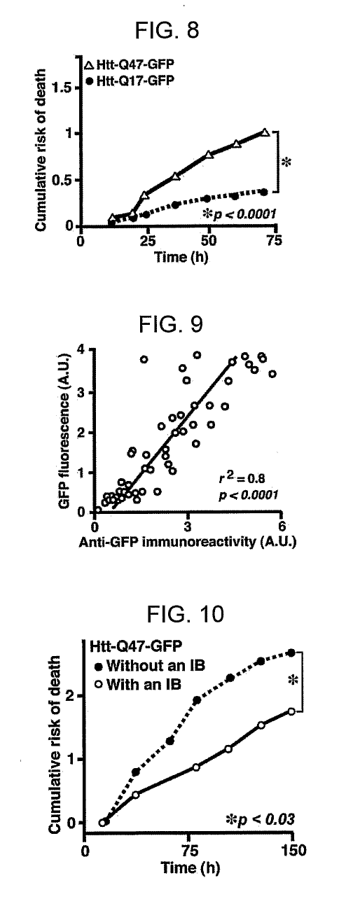

From the survival functions, hazard functions—the estimated instantaneous risk of death of individual cells, independent of population size—were deduced. Collett, D. Modeling survival data in medical research (ed. Collett, D.) (Chapman & Hall, London, 1994); and Therneau, T. M. Modeling survival data: Extending the Cox model (eds. Therneau, T. M. & Grambsch, P. M.) (Springer, N.Y., 2000). The cumulative risk of death was similar and remained relatively low in neurons transfected with GFP or htt^{ex1}-17Q-GFP (FIG. 1B; FIG. 8). However, htt^{ex1}-47Q-GFP, htt^{ex1}-72Q-GFP or htt^{ex1}-103Q-GFP significantly increased the risk, and the increase correlated with the length of the polyQ stretch. These results parallel features of HD: polyQ stretches longer than 35Q can cause neurodegeneration, with symptoms appearing sooner the longer the stretch. MacDonald, M. E. in Trinucleotide Diseases and Instability (ed. Oostra, B. A.) 47-75 (Springer-Verlag, Berlin, N.Y., 1998).

Knowing whether the risk of death changes over time can provide insights into the mechanisms responsible for neurodegeneration. Clarke et al. (2003) Nature 406:195-199. The cumulative risk of death increases as cells continually die (FIGS. 1A-D), but the risk of cell death does not necessarily change. To determine whether the risk of death changes, the linearity of the noncumulative hazard function was tested. A curved function means the risk of death changes over time; linearity indicates the risk is largely time-independent. The hazard functions for neurons transfected with htt^{ex1}-47Q-GFP, htt^{ex1}-72Q-GFP, or htt^{ex1}-103Q-GFP were essentially linear (F test, N. S.), indicating that the expanded polyQ stretches increase the risk of death relatively constantly over time.

However, these cultures contain subtypes of striatal neuron whose susceptibility varies in HD (Saudou et al. (1998) supra; Reiner et al. (1988) Proc. Natl. Acad. Sci. USA 85:5733-5737; and Richfield et al. (1995) Ann. Neurol. 38:852-861) and could mask a temporal variation in the risk of death conferred by polyQ expansion. Therefore, parallel experiments were performed in a homogenous, neuron-like pheochromocytoma 12 (PC12) cell line (FIG. 1C; FIG. 1D). PC 12 cells containing versions of htt with disease-associated polyQ expansions had a higher risk of death than those containing versions of htt with wild-type polyQ expansions, and the increase was relatively constant over time, as in primary striatal neurons. It was concluded that polyQ expansion beyond the disease threshold length leads to a steady but increased risk of cell death. These findings offer the first direct test and support of a recently proposed model of HD neurodegeneration inferred from pathological specimens. Clark et al. (2000) supra.

To examine IB formation and neuronal death, the ability to detect and monitor IBs in live neurons was confirmed. It was previously reported that in cultured striatal neurons, polyQ-expanded htt forms IBs that label with antibodies against ubiquitin (Saudou et al. (1998) supra), as in HD.

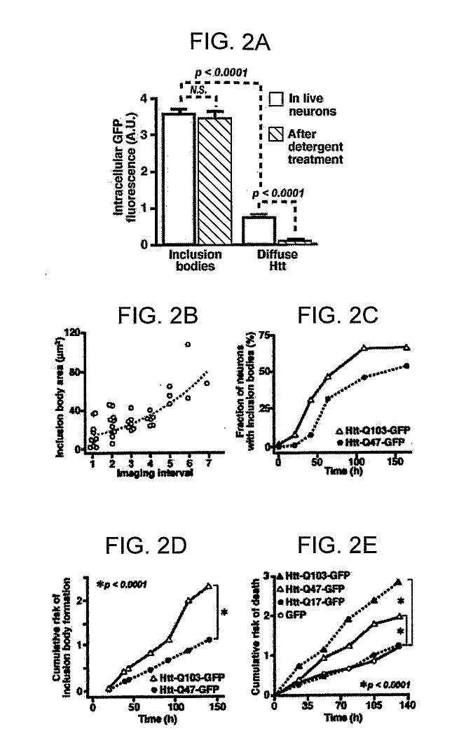

As with other cell types (Rajan et al. (2001) Proc. Natl. Acad. Sci. USA 98:13060-13065; and Moulder et al. (1999) J. Neurosci. 19:705-715), some neurons containing polyQ-expanded htt fused to GFP developed punctate, highly fluorescent intracellular structures resembling IBs. To further characterize these structures, GFP-tagged htt was fixed in situ, and its fluorescence was measured before and after detergent treatment. Kazantsev et al. (1999) supra. GFP fluorescence in the structures was not significantly affected, but was almost completely destroyed elsewhere in the neuron, suggesting that these structures were IBs (FIG. 2A).

Since the fluorescence intensity of htt^{ex1}-GFP within IBs is almost fivefold higher than that of diffuse htt^{ex1}-GFP elsewhere in the neuron, this distinction was used to identify IBs within living neurons and to follow their fates longitudinally. IBs formed in neurons transfected with htt^{ex1}-47Q-GFP, htt^{ex1}-72Q-GFP, or htt^{ex1}-103Q-GFP but not with htt^{ex1}-17Q-GFP or htt^{ex1}-25Q-GFP. IBs became detectable at <1 μm² and achieved sizes similar to those in HD, typically growing as long as the neuron remained alive (FIG. 2B). Larger IBs are also more common in later stages of HD. Gutekunst et al. (1999). J. Neurosci. 19:2522-2534. Thus, the size and behavior of IBs formed by htt in transfected striatal neurons resemble those seen in HD.

### Death Without IB Formation

If IBs trigger neuronal death through gradual sequestration and functional loss of other critical cellular proteins (Preisinger et al. (1999) Philos. Trans. R. Soc. Lond. B. Biol. Sci. 354:1029-1034), functional loss of these critical proteins—and therefore the risk of death-should increase with the number and size of IBs (i.e., IB load). Over time, the size of IBs (FIG. 2B) and the fraction of neurons (i.e., prevalence) that contain them (FIG. 2C) increased significantly. However, the risk of death from polyQ-expansion is relatively constant (see above), suggesting that IB load is unlikely to explain polyQ-dependent cell death.

The possibility that an earlier form of polyQ-expanded htt is the principal toxic species was considered. To test this possibility, the moment an IB was first detected (i.e., IB incidence) was recorded, and its relationship to polyQ-dependent death was measured. It has not been possible to measure IB incidence before because conventional approaches fail to record neurons that form IBs but die before they are detected and scored. IB incidence was over twofold greater for htt^{ex1}-103Q-GFP than for htt^{ex1}-47Q-GFP (FIG. 2D). Expansion from 47 to 103 glutamines had a larger effect on the incidence (FIG. 2D) than on the prevalence of IBs (FIG. 2d) because hazard analysis factors out confounding differences in survival between the two groups. Importantly, the polyQ expansion-dependent risk of death correlated better with initiation of IB formation than with IB load (FIG. 2D; FIG. 1B; FIG. 8). This finding suggests that the principal toxic species is an early IB intermediate or a form of diffuse intracellular htt.

Is IB formation even necessary for polyQ expansion-dependent death? IB formation has been dissociated from polyQ-dependent death (Saudou et al. (1998) supra; Klement et al. (1998) supra; Kuemmerle et al. (1999) Ann. Neurol. 46:842-849; Kim et al. (1999) J. Neurosci. 19:964-973), but the lack of longitudinal, single-cell analysis and the potential nonspecific effects of exogenous manipulations left the interpretation of these experiments in doubt. Perutz et al. (2001) Nature 412:143-144. For example, if IB formation accelerates death, neurons might die too rapidly to be detected. However, experiments in which images were collected every 2 h showed that only 1% of neurons that formed an IB within a 24 h interval also died within that period. In fact, most neurons that form IBs can be followed for at least 2 days (htt^{ex1}-47Q-GFP: 71%±4: htt^{ex1}-103Q-GFP: 55%±4). Thus, neurons that form IBs do not die too quickly to be detected. Moreover, survival analysis of htt-transfected neurons that do not form IBs showed an increased risk of death among neurons transfected with htt^{ex1}-47Q-GFP or htt^{ex1}-103Q-GFP but not htt^{ex1}-17Q-GFP (FIG. 2E). These findings indicate that IB formation is not required for polyQ expansion-dependent neuronal death and that other less aggregated or possibly monomeric species of polyQ-expanded htt are toxic.

Levels of Diffuse htt Govern Survival

If the principal toxic species of htt are distributed diffusely within neurons, their levels may be better predictors of neuronal death than IB formation. To determine if GFP fluorescence can be used to quantify protein levels in single cells (Hack et al. (2000) J. Neurosci. Methods 95:177-184), three experiments were performed.

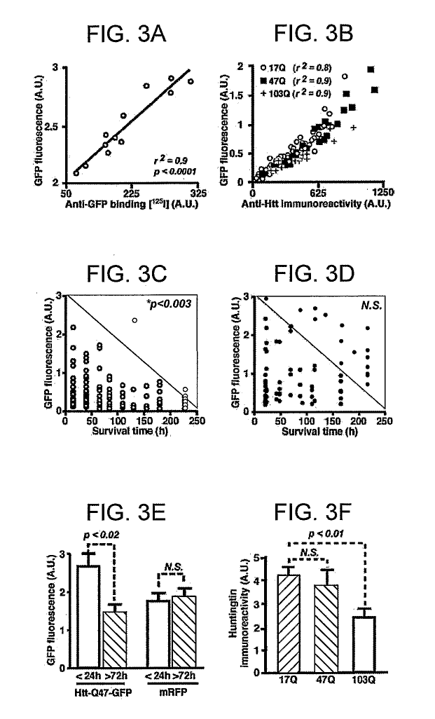

Both population-based and single-cell approaches showed that GFP fluorescence predicted the levels of GFP or of htt to which it was attached (FIG. 3A; FIG. 3B; FIG. 9). It was concluded that the amount of htt protein within living neurons can be quantified by imaging the fluorescence of the GFP tag.

To determine the relationship between htt levels and neuronal longevity, Cox proportional hazard analysis of neurons transfected with htt^{ex1}-47Q-GFP was used. The 47Q expansion is more typical among HD patients than 72Q or 103Q. Htt^{ex1}-47Q-GFP also leads to death more slowly than the longer expansions, increasing the ability to resolve relationships between its expression and survival or IB formation. Cox proportional hazard analysis was used because it can determine whether and to what extent levels of htt at an early time point within individual neurons can predict the longevities of those same neurons. The fluorescence from diffuse htt within neurons was measured, excluding IB fluorescence because htt within IBs might have different bioactivity. The levels of diffuse htt^{ex1}-47Q-GFP in neurons on the first day after transfection correlated significantly and negatively with lifespan (FIG. 3C). The amounts of GFP alone (FIG. 3D) or htt^{ex1}-17Q-GFP were not predictive. To exclude the possibility that neuron-subtype differences in vulnerability were required for the relationship that was observed, similar experiments were performed in the homogenous PC12 cell line. As in neurons, levels of htt^{ex1}-47Q-GFP on the first day of survival analysis were a significant and negative predictor of survival, whereas the expression of the co-transfected marker gene, mRFP, had no predictive value (FIG. 3e). These results suggest that more diffuse forms of polyQ-expanded htt are the principal toxic species and that their levels govern neuronal survival.

PolyQ expansions in ataxin-7 may cause toxicity by stabilizing ataxin-7, causing soluble forms to accumulate. Yoo et al. (2003) Neuron 37:383-401. Could a similar effect explain how levels of diffuse polyQ-expanded htt predict death? To avoid potential confounding effects of IB formation on htt levels, levels of diffuse htt in neurons were measured before IBs had formed. In contrast to findings with ataxin-7, polyQ expansion correlated with lower levels of diffuse htt^{ex1}-GFP (FIG. 3F); similar results have been reported in HD. Persichetti et al. (1996) Neurobiol. Dis. 3:183-190. Thus, the effects of polyQ expansion on htt levels do not explain polyQ expansion-dependent neuronal death. Rather, they indicate that the polyQ expansion confers toxicity on more diffuse forms of htt independently of its overall effect on the number of htt molecules.

IB Formation Prolongs Survival

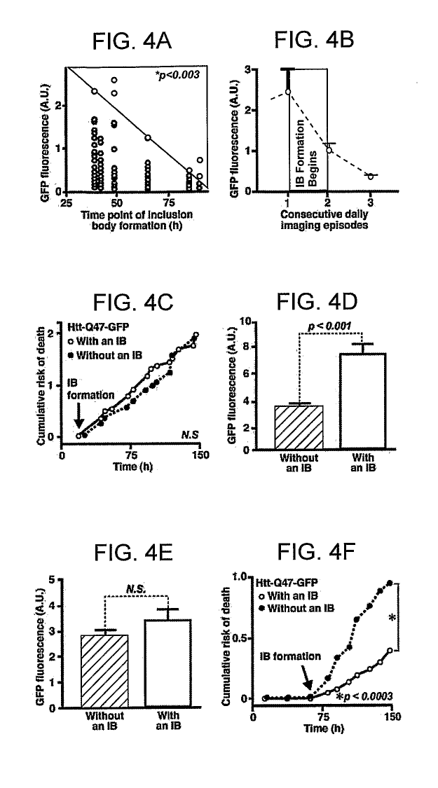

Correlations between polyQ expansion and IB, formation or neuronal death could suggest that IBs are pathogenic. Indeed, the levels of diffuse htt^{ex1}-47Q-GFP on day 1 after transfection correlated significantly and negatively with the time of IB formation (FIG. 4A). Thus, levels of diffuse htt^{ex1}-47Q-GFP predict whether and when an IB forms as well as longevity. However, the same patterns might be expected if IB formation were a cellular response to cope with more diffuse, toxic forms of htt. By analyzing images of neurons as they formed IBs, it was found that levels of diffuse htt-GFP elsewhere in the cell fell rapidly after an IB appeared, within a day or two, diffuse htt was nearly undetectable (FIG. 4B), and the rapid fall in diffuse GFP fluorescence directly correlated with the rapid growth of the IB. In a few cases, several days after diffuse htt was undetectable, the IB disappeared altogether.

To directly investigate the relationship between IB formation and the risk of death, the survival curves of neurons that did or did not develop IBs were compared. If IBs are pathogenic, neurons that develop them should die sooner than those that do not. If IB formation is beneficial, the reverse might be true, and if IB formation is incidental, there may be no correlation with survival. To avoid selection bias, all neurons that were alive at a particular time during the survival analysis were identified, that their fates followed prospectively. Neurons that contained or lacked an IB on the second day after transfection had similar risks of death (FIG. 4C).

However, upon closer examination, it was found that the subpopulation of neurons that form IBs on the second day also began with significantly higher intracellular levels of htt-GFP (FIG. 4D). Thus, although the survival curves of the two populations were indistinguishable, the survival of neurons that formed IBs was better than that predicted by the relatively high initial levels of htt-GFP expression (FIG. 3C). To test this idea further, the subpopulations of living neurons that either did or did not form an IB on the second day, and that had similar initial levels of htt-GFP, were identified. Prospective analysis revealed that neurons that formed IBs on the second day survived significantly longer than adjacent neurons that did not (FIG. 10).

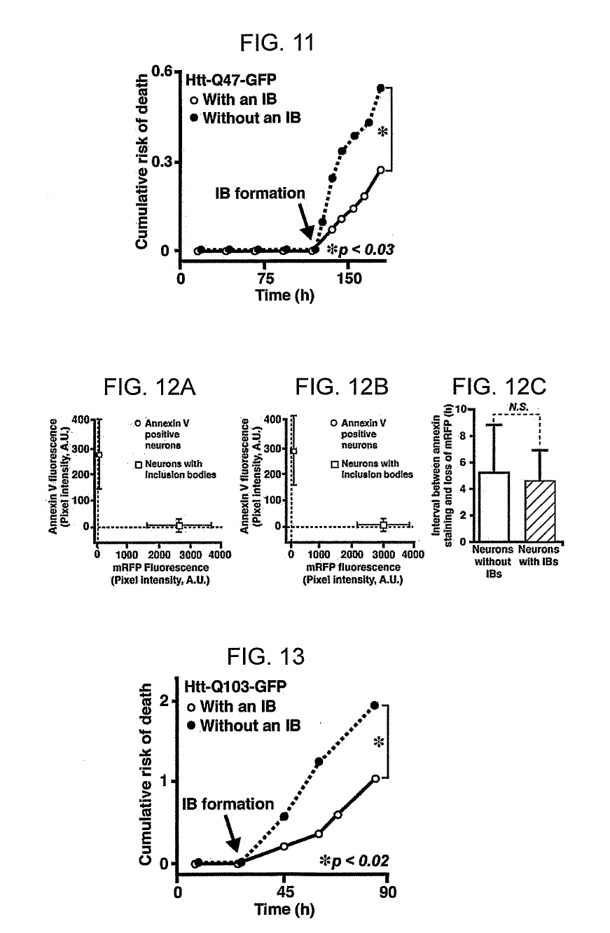

To further distinguish the contributions of htt-GFP expression and IB formation to neuronal survival, subpopulations of neurons with more closely matched levels of htt-GFP were compared. On either the fourth or sixth days after transfection, all living neurons started with similar levels of htt-GFP, irrespective of whether they had developed an IB (FIG. 4E). The survival of each of these populations was followed prospectively. Neurons that formed an IB on either the fourth or sixth days survived significantly longer than adjacent neurons, which were otherwise similar but without an IB. IB formation was associated with a drop in the cumulative risk of death (FIG. 4F, FIG. 11; FIG. 12A-C) to levels seen with wild-type htt (htt^{ex1}-17Q-GFP). Moreover, PC12 cells that formed IBs survived significantly longer than those that did not, indicating that neuron-subtype differences in IB formation and viability were not required for the relationship that was observed (FIG. 13). IB formation was generally associated with a reduction in more diffuse forms of intracellular htt and a corresponding improvement in survival.

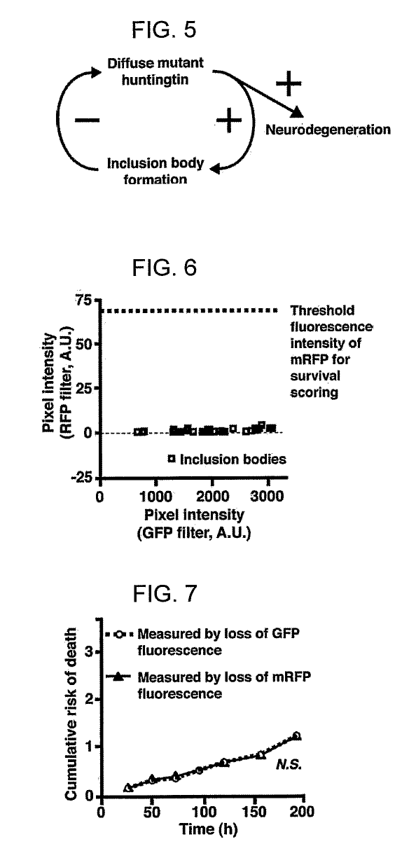

In this cellular model, IBs form in the cytoplasm and in the nucleus, as in HD. The nucleus appears to be an important site of toxicity for mutant htt. Saudou et al. (1998) supra; Kegel et al. (2002) J. Biol. Chem. 277:7466-7476; Peters et al. (1999) Mol. Cell. Neurosci. 14:121-128. Therefore, IBs could be pathogenic in one location and beneficial in another. Analysis of neurons with cytoplasmic or nuclear IBs showed similar survival curves for both populations, and both survived significantly longer than neurons without IBs. Thus, IB formation predicted increased survival regardless of the subcellular location. Together, the above-described findings indicate that IB formation protects neurons by reducing the levels of toxic diffuse forms of mutant htt (FIG. 5).

FIGS. 1A-D depict polyQ expansion-dependent cell death measured with an automated microscope. a, Survival analysis of neurons transfected with wild-type (htt^{ex1}-17Q-GFP) or mutant htt (htt^{ex1}-72Q-GFP) illustrates polyQ expansion-dependent death (n>100 neurons, 4 experiments). b, Hazard analysis demonstrates that versions of htt with disease-associated polyQ expansions increase the risk of death significantly and in a length-dependent fashion (n=4). c; d, Homogeneous PC12 cells that are either stably (c) or transiently (d) transfected with htt-GFP undergo a polyQ expansion-dependent decrease in survival and corresponding increase in death risk (n>200 PC12 cells, 2-3 experiments).

FIGS. 2A-E depict neuronal death without IB formation. a, Fluorescence intensity within IBs is very high, making it possible to monitor IBs in living neurons. (A.U.=arbitrary units of fluorescence intensity, n=10-541 neurons, 3 experiments). b, IB growth was measured daily (n=12). c, A cohort of neurons was monitored longitudinally. The fraction of neurons with IBs grows with time and is greater for those transfected with htt^{ex1}-103Q-GFP than htt^{ex1}-47Q-GFP (3experiments). e, Cumulative risk of IB formation is approximately twofold higher for htt^{ex1}-103Q-GFP than htt^{ex1}-47Q-GFP and parallels the cumulative risk curves for survival (n=680 neurons, 3 experiments). d, Neurons transfected with htt that do not form detectable IBs nevertheless exhibit a significant polyQ expansion-dependent increase in cumulative risk of death, indicating decreased survival. (n=480 neurons, 3 experiments).

FIGS. 3A-F depict the effect of diffuse mutant htt on neuronal survival. Levels of diffuse mutant htt protein predict death. a, Cellular GFP fluorescence correlates well with western blot measures of GFP within the same cells (n=2). b, Single-neuron levels of htt fused to GFP estimated by imaging GFP fluorescence correlate well with measurements by immunocytochemistry (n=2). c, Levels of diffuse htt^{ex1}-47Q-GFP are a significant and negative predictor of neuronal longevity. Fluorescence of diffuse htt^{ex1}-47Q-GFP was measured in individual neurons (n=217 neurons, 3 experiments) on the first day after transfection and plotted against their respective survival times. d, Levels of GFP alone do not correlate with neuronal survival (n=97 neurons, 3 experiments). e, Levels of htt^{ex1}-47Q-GFP but not the co-transfected marker, mRFP, are a significant and negative predictor of which PC12 cells live longer than 72 h (n=75). f, Mean levels of htt^{ex1}-GFP correlate significantly and negatively with the length of the polyQ stretch within htt^{ex1} (n>90 neurons, 3 experiments).

FIGS. 4A-F depict the effect of IB formation on neuronal survival. IB formation is associated with reduced intracellular levels of diffuse htt^{ex1}and improved neuronal survival. a, Levels of diffuse htt^{ex1}-47Q-GFP correlate with IB formation (n=105, 3 experiments). b, GFP fluorescence within single neurons was measured over a region adjacent to the site of IB formation. Upon IB formation, levels of htt^{ex1}-47Q-GFP elsewhere in the neuron fell rapidly (n=10). c, Neurons transfected with htt^{ex1}-47Q-GFP were divided into two cohorts depending on whether they contained an IB on the second day they were imaged. The risk of death and the overall survival of neurons in these two cohorts were not significantly different (n=193 neurons, 3 experiments). d, Neurons transfected with htt^{ex1}-47Q-GFP that contain an IB on the second day also begin with significantly higher levels of htt^{ex1}-47Q-GFP than the cohort of neurons without an IB on the second day. e, Neurons transfected with htt^{ex1}-47Q-GFP that form IBs on the fourth day begin with approximately the same levels of htt^{ex1}-47Q-GFP as the cohort of neurons that are alive on the fourth day but do not have IBs. f, IB formation is associated with reduced death risk and increased survival among neurons transfected with htt^{ex1}-47Q-GFP that are alive beginning on the fourth day (n=224 neurons, 3 experiments).

FIG. 6. Fluorescence of mRFP provides a measure of neuronal morphology and viability that is independent of htt^{ex1}-GFP. An independent measure is critical because the aggregation of polyQ-expanded htt into IBs can leave so little GFP fluorescence in the rest of the neuron that it can be difficult to determine whether that neuron is alive. Conversely, the GFP fluorescence of IBs is so bright that it might be detected with MRFP filters, possibly leading to erroneous measurements of neuronal survival. Therefore, the system was designed with a light source and appropriate fluorescence filters to permit observation of mRFP fluorescence from neurons independently of GFP fluorescence. To demonstrate that significant GFP fluorescence from bright IBs does not pass through the mRFP filter (potentially causing scoring errors), neurons were co-transfected with htt^{ex1}-Q72-GFP and blue fluorescent protein (BFP) and a subpopulation with the largest and brightest IBs (n=33) was identified. BFP was chosen to help identify neuronal morphology and viability independently of htt^{ex1}-GFP, the same way mRFP is normally used. However, the excitation and emission wavelengths for BFP are shorter than for GFP and much shorter than for mRFP, so there is no fluorescence in the RFP channel unless there is bleed through from GFP. Images of these cells were collected using either the GFP filter set or the mRFP filter set. Regions of the images corresponding to IBs (see FIG. 2a for details) were identified, and pixel intensities of these regions were measured and plotted against each other. If significant GFP fluorescence from bright IBs passes through mRFP filters, it should be detectable with mRFP filters, and it should correlate with the GFP fluorescence intensity of that IB. Instead, intensely fluorescent IBs detected with the GFP filter set were nearly nonfluorescent when measured with the mRFP filter set. Over a broad range of GFP intensities, there was no trend between the amount of GFP fluorescence in the IB and the amount of fluorescence measured from the corresponding region of the image with the mRFP filter set. By comparison, to be considered “positive” in this survival analysis, a pixel measured with the mRFP filter set must be over 20-fold higher (threshold fluorescence intensity) than these background levels. Consequently, there is no significant contamination of mRFP measurements by GFP fluorescence from bright IBs.

FIG. 7. Abrupt loss of fluorescence from transfected mRFP is a sensitive and specific assay of neuronal death. Striatal neurons were co-transfected with mRFP and GFP and low basal rates of neuronal death were observed with longitudinal imaging. Sequential images of the same field of neurons were collected with filter sets that detected fluorescence either from mRFP or from GFP. The longitudinal images of mRFP and GFP fluorescence were each subjected to survival analysis and the cumulative risk of death was plotted for each. The curves are nearly identical, indicating that the loss of fluorescence from either transfected mRFP or transfected GFP provides an equivalent measure of survival. If one of the two markers had detected death earlier than the other, the two curves would have diverged at an early time point and would have remained approximately parallel thereafter. (n>300 neurons, 2 experiments).

FIG. 8. PolyQ expansion-dependent cell death measured with an automated microscope. Hazard analysis demonstrates that versions of htt with disease-associated polyQ expansions (htt^{ex1}-Q47-GFP) increase the risk of death significantly compared with versions of htt that have wild-type polyQ expansions (htt^{ex1}-Q17-GFP). (n=100 neurons, 3 experiments).

FIG. 9. Single-neuron levels of GFP estimated by imaging correlate well with levels measured by immunocytochemistry (n>50, 4 experiments).

FIG. 10. Neurons with similar levels of htt^{ex1}-Q47-GFP that form IBs on the second day survive better than neurons that do not. Levels of htt^{ex1}-Q47-GFP were measured in neurons a day after transfection and the subpopulation that lived at least until the second day and had overlapping levels of htt^{ex1}-Q47-GFP (middle 50% of the range of htt-GFP levels) was monitored prospectively (n=125, 3 experiments).

FIG. 11. IB formation is associated with reduced death risk and increased survival among neurons transfected with htt^{ex1}-47Q-GFP that are alive beginning on the sixth day (n=140, 3 experiments).

FIGS. 12A-C. Neurons with IBs are viable as assessed by annexin V staining. To further evaluate the viability of neurons with IBs and the specificity and sensitivity of our system, the cells were stained with annexin V, a marker of early apoptosis. a, At 3 days after transfection, the two groups differed significantly in the mean fluorescence from annexin V and from MRFP (p<0.0001); error bars indicate SD. b, At 7 days after transfection, the remaining neurons gave almost identical results to those seen at 3 days after transfection p<0.0001). In these two examples, no neurons with an IB stained with annexin V. The result suggests that for any single time point, the vast majority of mRFP positive neurons with IBs have not initiated apoptosis and are viable (n=54 neurons). c, Loss of mRFP closely follows the appearance of annexin V staining and the interval is comparable in neurons with or without IBs. Striatal neurons were transfected with htt^{ex1}-Q47-GFP. Approximately 60 h after transfection, neurons were incubated with annexin V conjugated to Alexa Fluor 350 for 15 min and then subjected to automated imaging every 2 h. The medium (containing annexin V) was replaced every 4 h. On average, annexin V staining appeared about 5 h before mRFP fluorescence was lost, and the interval was not significantly different in neurons with and without IBs (n>49 neurons, 2 experiments).

FIG. 13. IB formation is associated with reduced death risk and increased survival among stably transfected PC12 cells that were induced to express htt^{ex1}-103Q-GFP and were alive beginning on the third day (n=41, 3 experiments). PC12 cells that formed an IB on the first (p<0.0001) or second p<0.03) day also survived significantly better than adjacent PC12 cells without IBs (n=91-200, 3 experiments).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for reducing a level of a toxic protein in the cytosol of a eukaryotic cell, the method comprising contacting the cell with an active agent that induces sequestration of the toxic protein into an inclusion body in the cell.

2. The method of claim 1, wherein the toxic protein is a polyglutamine expansion-containing protein.

3. The method of claim 1, wherein the toxic protein is selected from huntingtin, atropin-1, ataxin-1, ataxin-2, ataxin-3, ataxin-7, alpha 1A-voltage dependent calcium channel, and androgen receptor.

4. The method of claim 1, wherein the toxic protein is selected from a beta-amyloid polypeptide, a tau protein, a presenilin, an alpha-synuclein, and a prion protein.

5. The method of claim 1, wherein the toxic protein is a viral protein.

6. The method of claim 1, wherein the cell is selected from a neuronal cell, a muscle cell, and a cancerous cell.

7. A method of identifying an agent that induces sequestration of a toxic protein into an inclusion body, the method comprising:
a) contacting a cell with a test agent, wherein the cell comprises a toxic protein in the cytosol of the cell; and
b) determining the effect, if any, of the test agent on sequestration of the toxic protein into an inclusion body.

8. The method of claim 7, wherein the determining step comprises imaging the cell with an automated microscope system.

9. The method of claim 7, further comprising determining the effect, if any, of the test agent on viability of the cell.

10. A method of treating a disorder associated with the presence of a toxic protein in the cytosol of a cell, the method comprising administering to an individual having the disorder an effective amount of an agent that induces sequestration of the toxic protein into an inclusion body in the cell.

11. The method of claim 10, wherein the disorder is Huntington's Disease, and the toxic protein in a huntingtin protein comprising a polyglutamine expansion.

12. The method of claim 10, wherein the disorder is Alzheimer's Disease, and the toxic protein is β-amyloid precursor protein and/or β-amyloid protein.

13. The method of claim 10, wherein the disorder is a viral infection, and the toxic protein is a protein encoded by the virus.

14. A method of producing a protein of interest in a genetically modified host cell, the method comprising:
a) culturing the genetically modified host cell in the presence of an agent that induces sequestration of the recombinant protein into an inclusion body, wherein the genetically modified host cell comprises an expression vector comprising a nucleotide sequence encoding the protein of interest; and
b) purifying the protein of interest from the inclusion body.

15. The method of claim 14, wherein the purification step comprises:
a) disrupting the host cells, forming a cell lysate;
b) recovering the inclusion bodies from the cell lysate; and
c) solubilizing the recombinant protein present in the inclusion bodies.

16. The method of claim 15, further comprising re-folding the recombinant protein.

17. The method of claim 16, further comprising at least one additional purification step selected from high performance liquid chromatography, size exclusion chromatography, and affinity chromatography.
